(19) United States
(12) Patent Application Publication
(10) Pub. No.: US 2006/0178357 A1
(43) Pub. Date: Aug. 10, 2006

(54) Chphalosporin-derived mercaptans as inhibitors of serine and metallo-beta-lactamases

(76) Inventors: John D. Buynak, Dallas, TX (US); Hansong Chen, Germantown, MD (US)

Correspondence Address: JOHN BUYNAK, 10842 HAYFIELD DR, DALLAS, TX, 75238, US

(21) Appl. No.: 11/348,066

(22) Filed: Feb. 6, 2006

Related U.S. Application Data
(60) Provisional application No. 60/651,712, filed on Feb. 10, 2005.

(51) Int. Cl. A61K 31/545; C07D 501/14

(52) U.S. Cl. 514/200; 540/222

(57) ABSTRACT

Compounds of formula I: See Formula I in Figures Section
wherein R¹, R², R³, R⁴ and n have any of the values defined in the specification, and their pharmaceutically acceptable salts, are useful for inhibiting simultaneously serine and metallo-β-lactamase enzymes, for enhancing the activity of β-lactam antibiotics, and for treating β-lactam resistant bacterial infections in a mammal. The invention also provides pharmaceutical compositions, processes for preparing compounds of formula I, and novel intermediates useful for the synthesis of compounds of formula I.

### CROSS REFERENCE TO RELATED APPLICATIONS

This application claims the benefit of provisional patent application No. 60/651,712 filed Feb. 10, 2005.

### SPONSORED RESEARCH

There is no claim of rights to this invention by the sponsoring agencies.

### BACKGROUND OF THE INVENTION

The most commonly encountered mechanism of microbial resistance to the β-lactam antibiotics is bacterial production of β-lactamases, enzymes that hydrolytically destroy penicillins and cephalosporins. This type of resistance can be transferred horizontally by plasmids that are capable of rapidly spreading the resistance, not only to other members of the same strain, but even to other species. Due to such rapid gene transfer, a patient can become infected with different organisms, each possessing the same β-lactamase.

β-Lactamase enzymes have been organized into four molecular classes: A, B, C and D based on amino acid sequence. Classes A, C, and D are serine hydrolases, while class B are zinc metalloenzymes. The class B metallo-β-lactamases have an extraordinarily broad substrate profile, which includes not only typical β-lactamase substrates, such as the penicillins and cephalosporins, but also the carbapenems, which are usually stable to hydrolysis by the serine β-lactamases. Bacteria possessing these metalloenzymes have increased in clinical importance in recent years. Class B metalloenzymes are now responsible for resistance in a number of pathogenic bacteria including the Klebsiella, Serratia, Pseudomonas/Stenotrophomonas and Bacteroides genera. Multifocal outbreaks of class B metallo-β-lactamase-producing Pseudomonas aeruginosa, resistant to carbapenems and other broad-spectrum antibiotics, have been reported in Asian and in European hospitals. Many of these strains are known to possess two types of β-lactamase, and are therefore capable of combining the protective effects of both a metallo- and a serine-β-lactamase.

One strategy for overcoming this rapidly evolving bacterial resistance is the synthesis and administration of β-lactamase inhibitors. Frequently, β-lactamase inhibitors do not possess antibiotic activity themselves and are thus administered together with an antibiotic. One example of such a synergistic mixture is “AUGMENTIN” (a registered trademark of GlaxoSmithKline), which contains the antibiotic amoxicillin and the β-lactamase inhibitor, clavulanic acid.

Unfortunately, current commercial inhibitors target only class A serine β-lactamases, which have historically been the most clinically relevant. Clinically useful inhibitors of class B metallo-β-lactamases are, at present, nonexistent. The current application describes compounds that simultaneously inhibit both class B metallo-β-lactamases and also class C serine β-lactamases. Such compounds have commercial and humanitarian significance.

This invention acknowledges the partial support of the Robert A. Welch Foundation, Grant N-0871, the Texas Higher Education Coordinating Board, and the National Institutes of Health, all of whom claim no rights.

### SUMMARY OF THE INVENTION

The present invention provides unique cephalosporin derivatives as new compositions of matter that are simultaneously potent inhibitors of both the metallo-β-lactamases as well as one or more of the serine-β-lactamases. Accordingly, the invention provides a compound of formula (I):

### See Formula I (in Figures Section)

wherein

R¹ is hydrogen, (C₁-C₁₀)alkanoyl C(═O)R, (C₁-C₁₀)alkoxycarbonyl C(═O)OR, (C₁-C₁₀)carboxamido (C═O)NHR, (C₁-C₁₀)alkyl, (C₃-C₈)cycloalkyl, (C₂-C₁₀)alkenyl, (C₂-C₁₀)alkynyl, aryl, heteroaryl, oxazolidinyl, isoxazolidinyl, or morpholinyl;

R² is hydrogen, (C₁-C₁₀)alkyl, (C₃-C₈)cycloalkyl, (C₂-C₁₀)alkenyl, (C₂-C₁₀)alkynyl, —COOR, —CONRR, cyano, —C(═O)R, aryl, heteroaryl, oxazolidinyl, isoxazolidinyl, morpholinyl, —S(O)ₙR, —NRR, azido, or halo;

R³ is acetoxymethyl (CH₂OAc), carbamoyloxymethyl (CH₂O(C═O)NH₂), hydrogen, methyl, halo, (C2-C10)alkyl, cyano, vinyl (—CH═CH2), (C1-C10)substituted vinyl (—CH═CHR), (C1-C10)alkanoylvinyl (—CH═CHCOR), (C1-C10)alkoxycarbonylvinyl (—CH═CHCO2R), (C1-C10)carboxaminovinyl (—CH═CHCONHR), (C2-C10)alkynyl, (C1-C10)alkanoyl, (C3-C8)cycloalkyl, aryl, heteroaryl, (C1-C10)substituted aryl, (C1-C10)alkyl, heteroaryl(C1-C10)alkyl, S(O)ₙR wherein n=0, 1, or 2, corresponding to the C3 sidechain sulfide, sulfoxide and sulfone and R is (C1-C10)alkyl, aryl, substituted aryl, heteroaryl, substituted heteroaryl.

R⁴ is hydrogen, acyloxymethyl (CH₂O(C═O)R), (C₁-C₁₀)alkyl, (C₃-C₈)cycloalkyl, (C₂-C₁₀)alkenyl, (C₂-C₁₀)alkynyl, aryl, or heteroaryl; or a pharmaceutically acceptable salt thereof, especially including sodium, potassium, and lithium.

n is 0, 1, or 2;

### BRIEF DESCRIPTION OF THE FIGURES

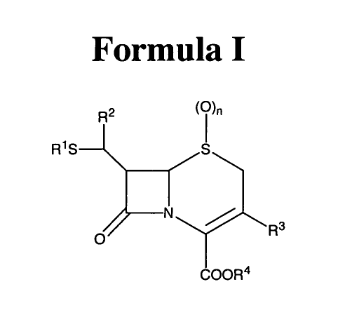

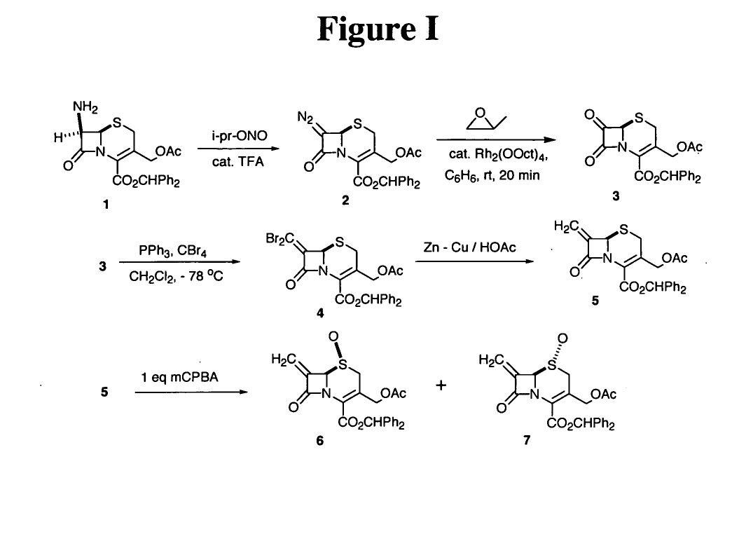

FIGS. 1, 2, 3, 4, and 5 illustrate the preparation of compounds of the invention (shown in the Figures section)

FIG. 1

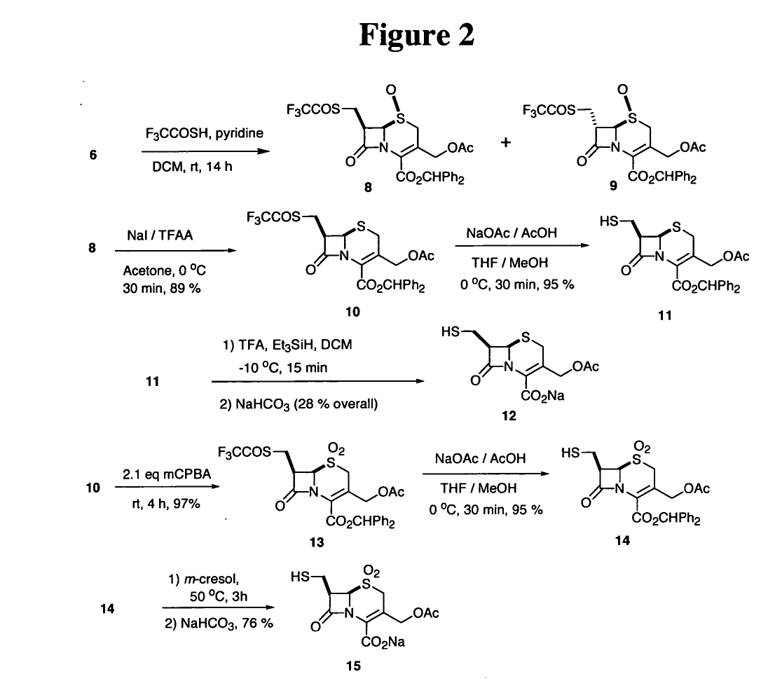

FIG. 2

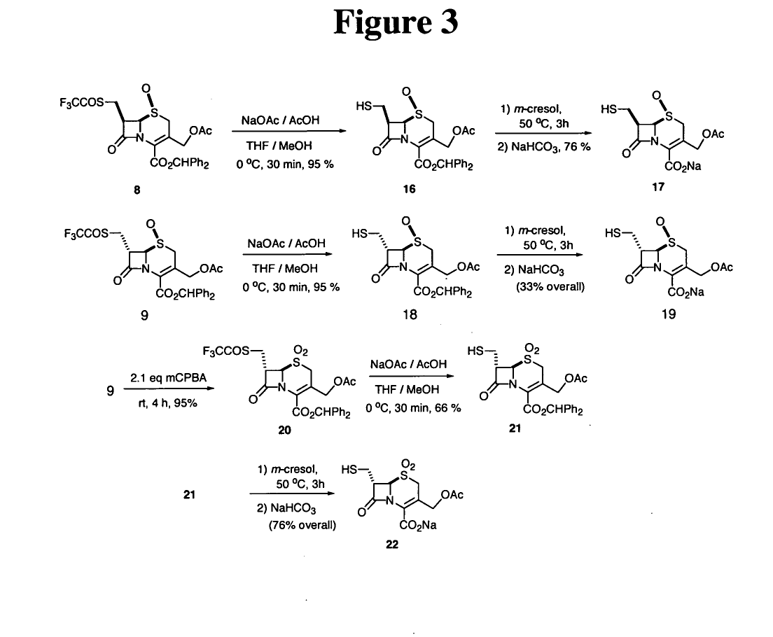

FIG. 3

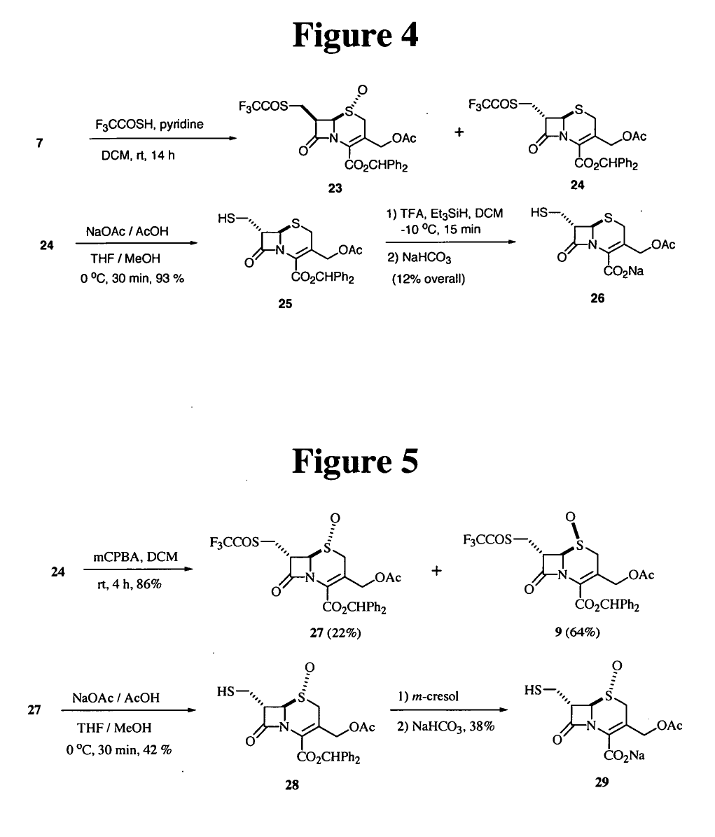

FIG. 4

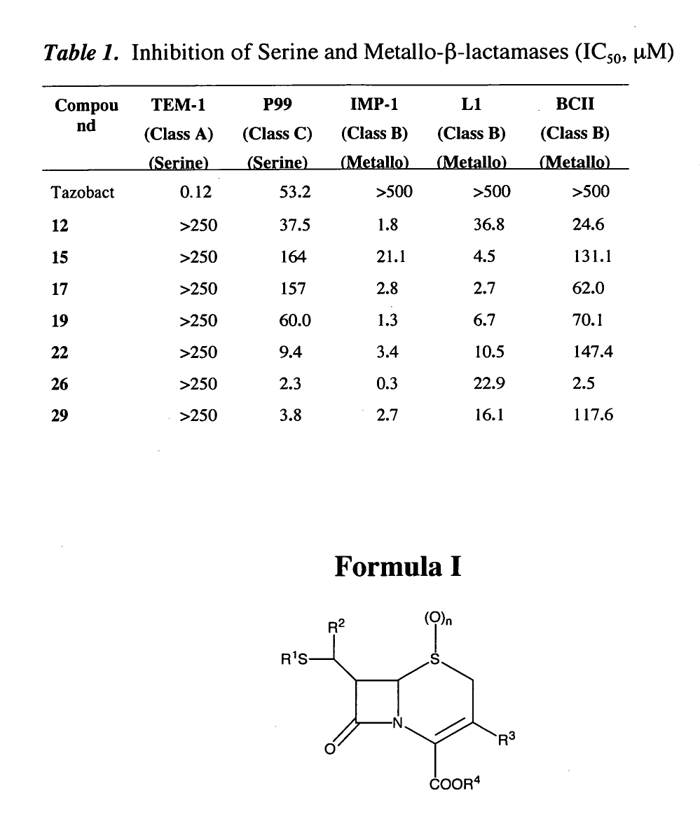

FIG. 5

### DETAILED DESCRIPTION OF THE INVENTION

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein each X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having one or more chiral centers may exist and be isolated as optically active and racemic forms. Some compounds may exhibit polymorphism. It is to be understood that the present invention encompasses any racemic, optically-active, polymorphic, or stereoisomeric form, or mixtures thereof, of a compound of the invention, that possesses the useful properties described herein, it being well known in the art how to prepare optically active forms (for example, by resolution of the racemic form by recrystallization techniques, by synthesis, from optically-active starting materials, by chiral synthesis, or by chromatographic separation using a chiral stationary phase) and how to determine β-lactamase inhibitory activity using the tests described herein, or using other tests which are well known in the art. Preferably, the absolute stereochemistry of compounds of the invention is that shown in formula I.

Specific and preferred values listed below for radicals, substituents, and ranges, are for illustration only and they do not exclude other defined values or other values within defined ranges for the radicals and substituents.

Specifically, (C₁-C₁₀)alkyl can be methyl, ethyl, propyl, isopropyl, butyl, iso-butyl, sec-butyl, pentyl, 3-pentyl, hexyl, heptyl, octyl, nonyl or decyl; (C₃-C₈)cycloalkyl can be cyclopropyl, cyclobutyl, cyclopentyl, cyclohexyl, cycloheptyl, or cyclooctyl; (C₁-C₁₀)alkoxy can be methoxy, ethoxy, propoxy, isopropoxy, butoxy, iso-butoxy, sec-butoxy, pentoxy, 3-pentoxy, hexyloxy, heptyloxy, octyloxy, nonyloxy, or decyloxy; (C₂-C₁₀)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, or 9-decenyl; (C₂-C₁₀)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, or 9-decynyl; (C₁-C₁₀)alkanoyl can be acetyl, propanoyl, butanoyl, isobutanoyl, pentanoyl, hexanoyl, heptanoyl, octanoyl, nonanoyl, or decanoyl; and (C₂-C₁₀)alkanoyloxy can be acetoxy, propanoyloxy, butanoyloxy, isobutanoyloxy, pentanoyloxy, hexanoyloxy, heptanoyloxy, octanoyloxy, nonanoyloxy, or decanoyloxy. Specifically “aryl” can be phenyl, indenyl, or naphthyl. Specifically, “heteroaryl” can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide), thiadiazolyl, thiatriazolyl, oxadiazolyl, or quinolyl (or its N-oxide). More specifically, “heteroaryl” can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide). More specifically, heteroaryl can be pyridyl.

A specific value for R³ is methyl, acetoxymethyl, and carbamoyloxymethyl.

A preferred value for R¹, R², and R⁴ is hydrogen; and for R³ is methyl.

Another preferred value for R⁴ is a suitable metal salt, including sodium, potassium, and lithium.

Another preferred value for R⁴ is acyloxymethyl (−CH2-OC(O)R)

Another preferred value for R² is methyl.

Another preferred value for R² is benzyl (PhCH₂).

Another preferred value for R² is pyridyl (e.g. 2-pyridyl).

Another preferred value for R³ is vinyl or substituted vinyl.

Another preferred value for R³ is C1-C10 thioalkyl (S(O)ₙR), where n=0, 1, or 2.

Another preferred value for R³ is thioaryl (S(O)ₙAr), where n=0, 1, or 2.

Another preferred value for R³ thioheteroaryl (S(O)ₙHet), where n=0, 1, or 2.

Processes and novel intermediates useful for preparing compounds of formula I are provided as further embodiments of the invention and are illustrated by the following procedures in which the meanings of the generic radicals are as given above unless otherwise qualified. Certain compounds of formula (I) are also useful as intermediates for preparing other compounds of formula (I).

A compound of formula I wherein n is 1 can be prepared by oxidation of suitably protected intermediates wherein n is 0, using one equivalent of an acceptable oxidizing agent, for example, mCPBA. For example, oxidation of compound 5 (FIG. 1) or compound 24 (FIG. 5) would produce selective oxidation of the dihydrothiazine sulfur, thus leading to intermediates suitable for the preparation of formula I (n=1).

A compound of formula I wherein R⁴ is a metal salt can generally be prepared from a corresponding ester of formula I wherein R⁴ is other than a metal salt by hydrolysis, using techniques which are well known in the art, as illustrated in FIG. 2 for the conversion of a compound of formula 11 to a compound of formula 12.

It is noted that many of the starting materials employed in the synthetic methods described above are commercially available or are reported in the scientific literature. It is also noted that it may be desirable to optionally use a protecting group during all or portions of the above described synthetic procedures. Such protecting groups and methods for their introduction and removal are well known in the art (see Greene, T. W.; Wutz, P. G. M. “Protecting Groups In Organic Synthesis” second edition, 1991, New York, John Wiley & sons, Inc.).

Compounds of Formula I can be prepared as illustrated in FIGS. 2, 3, 4, and 5. Thus, as shown in FIG. 1, the readily available benzhydryl ester of 7-aminocephalosporanic acid (7-ACA), 1, can be converted to benzhydryl 7-oxocephalosporanate (3) utilizing the method of J. D. Buynak (J. D. Buynak et. al., Tetrahedron Letters 1999, 39, 4945-4946). Treatment of ketone 3 with a mixture of carbon tetrabromide and triphenylphosphine produces dibromide 4, which can be dehalogenated with commercially available zinc/copper couple to produce terminal alkene 5. The cephalosporin is then oxidized with mCPBA to produce a separable mixture of sulfoxides 6 and 7.

As illustrated in FIGS. 2 and 4, each of these two isomeric sulfoxides can be individually treated with trifluorothioacetic acid to produce a mixture of two separable trifluoroacetates (total four compounds resulting from the two sulfoxides). As illustrated in FIGS. 2, 3, 4, and 5, each of these four trifluoroacetates can be transformed to three separate inhibitors through one of three procedures:

Proceedure 1. To form Cephalosporin Sufoxides (inhibitors 17, 19, and 29). a) removal of the trifluoroacetyl protecting group by treatment with sodium acetate/acetic acid in methanol; b) removal of the benzhydryl ester by treatment with trifluoroacetic acid in the presence of triethylsilane

Procedure 2: To form Cephalosporin Sulfones (Inhibitors 15 and 22): a) oxidizing the sufoxide to the corresponding sulfone with mCPBA, b) removal of the trifluoroacetyl protecting group by treatment with sodium acetate/acetic acid in methanol, and c) removal of the benzhydryl ester by treatment with trifluoroacetic acid in the presence of triethylsilane.

Procedure 3: To form Cephalosporin Sulfides (Inhibitors 12 and 26): a) reducing the sulfoxide to the corresponding sulfide by treatment with sodium iodide and trifluoroacetic anhydride (TFAA), b) removal of the trifluoroacetyl protecting group by treatment with sodium acetate/acetic acid in methanol, and c) removal of the benzhydryl ester by treatment with trifluoroacetic acid in the presence of triethylsilane.

Each of these final products was evaluated against representative serine- and metallo-β-lactamases. The results are shown in Table 1.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

Preferred salts of the invention include disalts prepared from acids of formula (I) wherein R¹ or R² is carboxy and R⁴ is hydrogen. Preferred salts also include monosalts (e.g. a sodium salt) prepared from an acid of formula (I) wherein R⁴ is hydrogen. The invention also provides a method for preparing a compound of the invention comprising forming a mono-, di-, or tri-salt from a coresponding compound of formula (I).

The compounds of formula I can be formulated as pharmaceutical compositions and administered to a mammalian host, such as a human patient in a variety of forms adapted to a selected route of administration, i.e., by oral, parenteral, intravenous, intramuscular, topical, or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are disclosed in Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. Single dosages for injection, infusion or ingestion will generally vary between 50-1500 mg, and may be administered, i.e., 1-3 times daily, to yield levels of about 0.5-50 mg/kg, for adults.

Accordingly, the invention provides a pharmaceutical composition, comprising an effective amount of a compound of formula I as described hereinabove; or a pharmaceutically acceptable salt thereof; and a pharmaceutically acceptable carrier. The invention also provides a pharmaceutical composition comprising an effective amount of a compound of formula I, as described hereinabove; or a pharmaceutically acceptable salt thereof; a β-lactam antibiotic; and a pharmaceutically acceptable carrier. Any β-lactam antibiotic is suitable for use in the pharmaceutical composition of the invention. β-Lactam antibiotics which are well known in the art include those disclosed by R. B. Morin and M. Gorin, M. Eds.; Academic Press, New York, 1982; vol. 1-3. Preferred β-lactam antibiotics, suitable for use in the pharmaceutical composition of the invention, include β-lactam antibiotics which are preferentially deactivated by Class A and Class C β-lactamase enzymes, for example, amoxicillin, piperacillin, ampicillin, ceftizoxime, cefotaxime, cefuroxime, cephalexin, cefaclor, cephaloridine, and ceftazidime.

The ability of a compound of the invention to function as a β-lactamase inhibitor can be demonstrated using the test described below, or using other tests which are well known in the art.

Representative compounds of the invention were evaluated as inhibitors of two serine β-lactamases: Enterobacter cloacae P99 (class C), TEM-1 (class A), as well as two class B enzymes: the L1 metallo-β-lactamse derived from Stenotrophomonas maltophilia) and the metallo-β-lactamase derived from the Bacillus cereus microorganism. The IC₅₀ value of each compound was determined as follows. Assay method involves 4 min incubation of a solution of inhibitor and enzyme (0.1 to 1 μM in enzyme), followed by transfer of an aliquot into a dilute solution of the substrate nitrocefin. Hydrolysis is monitored spectrophotometrically at 480 nm for 1 min. The rate is constant throughout this period. Error is ±10%, based on multiple experiments.

The results are summarized in Table 1. The relative inhibition activity of both the serine and metallo-β-lactamases by compounds 12, 15, 17, 19, 22, 26, and 29 of the invention is contrasted with that of tazobactam a commercial β-lactamase inhibitor. In particular, all of the former 6-(mercaptomethyl)cephalosporinates of this invention possess the ability to inhibit the class B β-lactamases, while tazobactam does not possess such ability. In addition, selected 6-(mercaptomethyl)cephalosporinates, especially including 22, 26, and 29 are superior to tazobactam against a representative class C β-lactamase.

### Table I (see Figures Section)

Compounds of the invention have been shown to possess activity as inhibitors of both the serine and metallo-β-lactamases. Accordingly, the invention provides a method comprising inhibiting both serine and metallo-β-lactamases by contacting said enzymes with an effective amount of a compound of formula I; or a pharmaceutically acceptable salt thereof. The β-lactamases may be contacted with the compound of claim 1 in vitro or in vivo. The invention also provides a therapeutic method comprising inhibiting both serine and metallo-β-lactamases in a mammal (preferably a human) in need of such therapy, by administering an effective inhibitory amount of a compound of formula I; or a pharmaceutically acceptable salt thereof.

Because compounds of the invention inhibit both the serine and metallo-β-lactamase enzymes, they may also be useful to increase the effectiveness of β-lactam antibiotics which are degraded by such enzymes. Accordingly, the invention provides a method comprising enhancing (increasing by a detectable amount ) the activity of a β-lactam antibiotic, by administering the β-lactam antibiotic to a mammal (preferably a human) in need thereof, in combination with an effective amount of a compound of formula I; or a pharmaceutically acceptable salt thereof.

The invention also provides a method comprising treating a β-lactam resistant bacterial infection in a mammal (preferably a human), by administering an effective amount of a β-lactam antibiotic in combination with an effective serine and metallo-β-lactamase inhibiting amount of a compound of formula I; or a pharmaceutically acceptable salt thereof.

Additionally, the invention provides a compound of formula I for use in medical therapy (preferably for use in treating a β-lactam resistant infection), as well as the use of a compound of formula I for the manufacture of a medicament useful for reducing both serine and metallo-β-lactamase activity in a mammal.

Compounds of the invention possess specific 6-(mercaptomethyl) substituents and specific 2′-substituents. In particular, the 6-mercaptomethyl substituent conveys upon this class of molecules the capability to act as inhibitors of the class B metallo-β-lactamases, while maintaining inhibition of class C serine-β-lactamases. Additionally, compounds of the invention may possess other biological or pharmacological properties which make them superior to known compounds as therapeutic agents.

The invention will now be illustrated by the following non-limiting examples.

### EXAMPLE 1

Benzhydryl 7-diazocephalosporanate (2). To a solution of benzhydryl 7-aminocephalosporate (30 g, 68.49 mmol) in ethyl acetate (200 mL) were added isopropyl nitrite (45 mL) and TFA (0.20 mL) at room temperature. The mixture was stirred at room temperature for 10 min (The reaction was monitored by TLC). The volatiles were removed at reduced pressure to give the yellow solid. The product was used directly in the next reaction without further purification.

### EXAMPLE 2

Benzhydryl 7-oxocephalosporanate (3). To a solution of benzhydryl 7-diazocephalosporate (3) (68.49 mmol) in dry benzene (180 mL) and propylene oxide (240 mL) was added rhodium octanoate dimer (0.24 g), the mixture was stirred at room temperature for 15 min (evolution of gas was observed immediately after the addition of the catalyst). Volatiles were removed at reduced pressure to produce a brown solid. The crude product was directly used in next reaction without further purification.

### EXAMPLE 3

Benzhydryl 7-(dibromomethylene)cephalosporanate (4). To a solution of Ph₃P (64.6 g, 123.29 mmol) in anhyd CH₂Cl₂(400 mL) was added CBr₄ (40.93 g, 123.29 mmol) in one portion at 0° C. under an argon atmosphere. The mixture was stirred at room temperature for 30 min. The mixture was cooled to −78° C. and a cold (−78° C.) solution of benzhydryl 7-oxocephalosparate (68.49 mmol) in anhyd CH₂Cl₂ (100 mL) was added. After stirring at −78° C for 1 h, it was concentrated and purified by flash column chromatography on silica gel (100% CH₂Cl₂) to give pure product (20%). IR (neat, cm⁻¹) 1777, 1739; ¹H NMR (400 MHz, CDCl₃) δ 7.46-7.24 (m, 10H, Ar), 6.95 (s, 1H, CHPh₂), 5.14 (s, 1H, C6 CH), 4.93 (d, J=13.5 Hz, 1H, CH₂OAc), 4.69 (d, J=13.5 Hz, 1H, CH₂OAc), 3.46 (d, J=18.3 Hz, 1H, C2 CH), 3.28 (d, J=18.3 Hz, 1H, C2 CH), 1.97 (s, 3H, Ac); ¹³C NMR (100 MHz, CDCl₃) δ 170.2, 160.5, 155.6, 142.4, 139.0, 138.8, 128.4, 128.3, 128.0, 127.9, 127.5, 126.9, 125.1, 92.7, 79.8, 62.9, 60.0, 26.9, 20.5.

### EXAMPLE 4

Benzhydryl 7-methylenecephalosporanate (5). To a hot solution of copper (II) acetate (7.0 g, 38.54 mmol) in acetic acid (120 mL) at 70° C. was added zinc dust (70 g, 1.10 mol) slowly. After the addition, the mixture was stirred at the same temperature for 5 min. Then the mixture was cooled to room temperature, the solution of dibromide (14 g, 23.6 mmol) in acetic acid was added. After stirring at room temperature for 4 h, the mixture was filtered through a pad of celite and evaporated to dryness. The residue was dissolved in CH₂Cl₂ (500 mL) and the resulting solution was washed with water and brine, dried over Na₂SO₄ and evaporated. Further purification by flash column chromatography on silica gel (1% EtOAc/CH₂Cl₂ to 8% EtOAc/CH₂Cl₂) produced pure product (6.2 g, 60%). IR (neat, cm⁻¹) 1774, 1732, 1717; ¹H NMR (400 MHz, CDCl₃) δ 7.48-7.27 (m, 10H, Ar), 6.99 (s, 1H, CHPh₂), 6.00 (t, J=1.7 Hz, 1H, alkene), 5.56 (d, J=1.1 Hz, 1H, alkene), 5.14 (s, 1H, C6 CH), 4.95 (d, J=13.3 Hz, 1H, CH₂OAc), 4.71 (1, J=13.3 Hz, 1H, CH₂OAc), 3.54 (d, J=18.3 Hz, 1H, C2 CH), 3.35 (d, J=18.3 Hz, 1H, C2 CH), 2.01 (s, 3H, Ac); ¹³C NMR (100 MHz, CDCl₃) δ 170.3, 160.8, 158.9, 146.0, 139.1, 138.9, 128.4, 128.3, 128.0, 127.9, 127.6, 126.9, 122.4, 114.8, 79.7, 63.0, 56.6, 27.9, 20.5.

### EXAMPLE 5

Benzhydryl 7-methylenecephalosporanate-1β-oxide (6) and Benzhydryl 7-methylenecephalosporanate-1α-oxide (7). To a solution of terminal alkene sulfide (17.85 g, 40.75 mmol) in CH₂Cl₂ (250 mL) at 0° C. was added mCPBA (10.04 g, 40.75 mmol) in several portions. The reaction mixture was stirred for 2 h at room temperature. The mixture was diluted with CH₂Cl₂, then washed with aq sodium metabisulfite solution, saturated NaHCO₃ solution and brine, dried over Na₂SO₄, evaporated, the residue was purified by flash column chromatography on silica gel (15% EtOAc/CH₂Cl₂ to 80% EtOAc/CH₂Cl₂) to give β isomer (7.5 g, 41%) and α isomer (5.0 g, 27%) (ratio 6:4).

7 α isomer (top spot): IR (neat, cm⁻¹) 1783, 1736; ¹H NMR (400 MHz, CDCl₃) δ 7.46-7.28 (m, 10H, Ar), 6.97 (s, 1H, CHPh₂), 6.18 (t, J=1.80 Hz, 1H, alkene), 5.56 (d, J=1.3 Hz, 1H, alkene), 5.00 (d, J=13.9 Hz, 1H, CH₂OAc), 4.86 (s, 1H, C6 CH), 4.74 (d, J=13.9 Hz, 1H, CH₂OAc), 4.00 (d, J=17.1 Hz, 1H, C2 CH), 3.54 (d, J=17.1 Hz, 1H, C2 CH), 2.05 (s, 3H, Ac); ¹³C NMR (100 MHz, CDCl₃) δ 170.2, 159.7, 157.4, 143.4, 138.8, 138.7, 128.6, 128.5, 128.3, 128.2, 127.7, 127.1, 126.9, 122.4, 118.1, 80.4, 73.4, 62.3, 50.7, 20.6.

6 β isomer (bottom spot): IR (neat, cm⁻¹) 1779, 1735; ¹H NMR (400 MHz, CDCl₃) δ 7.50-7.27 (m, 10H, Ar), 7.00 (s, 1H, CHPh₂), 6.19 (d, J=1.4 Hz, 1H, alkene), 5.72 (t, J=1.5 Hz, 1H, alkene), 5.18 (d, J=13.7 Hz, 1H, CH₂OAc), 4.79 (s, 1H, C6 CH), 4.65 (d, J=13.7 Hz, 1H, CH₂OAc), 3.85 (d, J=18.4, 1H, C2 CH), 3.22 (d, J=18.4 Hz, 1H, C2 CH), 2.01 (s, 3H, Ac); ¹³C NMR (100 MHz, CDCl₃) δ 170.3, 159.9, 158.2, 141.5, 139.0, 138.8, 128.4, 128.3, 128.1, 128.0, 127.6, 127.0, 126.8, 117.3, 116.2, 80.2, 68.9, 63.4, 46.7, 20.6.

### EXAMPLE 6

Benzhydryl 7β-(trifluoroacetylsulfanylmethyl)cephalosporanate-1β-oxide (8) and Benzhydryl 7α-(trifluoroacetylsulfanylmethyl)cephalosporanate-1β-oxide (9). To a solution of 6 (3.5 g, 7.71 mmol) in anhyd CH₂Cl₂ (50 mL) was added trifluorothioacetic acid (2.86 mL, 30.84 mmol) under argon. After the mixture was cooled to 0° C., pyridine (0.80 g, 0.82 mL, 10.18 mmol) was added slowly. The reaction was stirred at rt overnight. The volatiles were removed under reduced pressure, the residue was purified by flash column chromatography on silica gel (10% EtOAc/CH₂Cl₂to 50% EtOAc/CH₂Cl₂) to give the 7β isomer, 8, in 42% yield and the 7α isomer, 9, isomer in 28% yield.

### EXAMPLE 7

Benzhydryl 7β-(trifluoroacetylsulfanylmethyl)cephalosporanate-1β-oxide (8) (top spot). IR (neat, cm⁻¹) 1785, 1738, 1716; ¹H NMR (400 MHz, CDCl₃) δ 7.48-7.28 (m, 10H, Ar), 6.97 (s, 1H, CHPh₂), 5.22 (d, J=13.9 Hz, 1H, CH₂OAc), 4.67 (d, J=13.9 Hz, 1H, CH₂OAc), 4.34 (dd, J₁=5.2 Hz, J₂=1.4 Hz, 1H, C6 CH), 4.12 (m, 1H, C7 CH), 3.85-3.64 (m, 2H, SCH₂), 3.83 (d, J=18.6 Hz, 1H, C2 CH), 3.26 (d, J=18.6 Hz, 1H, C2 CH), 2.02 (s, 3H, Ac); ¹³C NMR (100 MHz, CDCl₃) δ 184.6 (q, J=36.4 Hz, CF₃CO), 170.4, 162.6, 159.6, 138.9, 138.8, 128.6, 128.5, 128.3, 128.1, 127.7, 127.0, 125.8, 118.2, 115.3 (q, J=289 Hz, CF₃), 80.4, 64.6, 63.3, 53.3, 46.6, 23.0, 20.6.

### EXAMPLE 8

Benzhydryl 7α-(trifluoroacetylsulfanylmethyl)cephalosporanate-1β-oxide (9) (bottom spot). IR (neat, cm⁻¹) 1786, 1736, 1713; ¹H NMR (400 MHz, CDCl₃) δ 7.46-7.30 (m, 10H, Ar), 6.98 (s, 1H, CHPh₂), 5.13 (d, J=13.17 Hz, 1H, CH₂OAc), 4.63 (d, J=13.17 Hz, 1H, CH₂OAc), 4.26 (br s, 1H, C6 CH), 3.95 (m, 1H, C7 CH), 3.80 (d, J=18.6 Hz, 1H, C2 CH), 3.59 (m, 2H, SCH₂), 3.28 (d, J=18.6 Hz, 1H, C2 CH), 2.01 (s, 3H, Ac); ¹³C NMR (100 MHz, CDCl₃) δ 184.0 (q, J=40.7 Hz, CF₃CO), 170.2, 161.7, 159.7, 138.9, 138.7, 128.4, 128.2, 128.0, 128.0, 127.4, 126.7, 125.6, 117.4, 115.2 (q, J=288.5 Hz, CF₃), 80.0, 62.8, 51.7, 25.4, 20.3.

### EXAMPLE 9

Benzhydryl 7β-(trifluoroacetylsulfanylmethyl)cephalosporanate (10). To a solution of benzhydryl 7β-(trifluoroacetylsulfanylmethyl)cephalosporanate-1β-oxide (8) (0.67 g, 1.15 mmol) and sodium iodide (0.86 g, 5.76 mmol) in dry acetone (12 mL) at 0° C. was added trifluoroacetic anhydride (1.35 g, 0.91 mL, 6.45 mmol) under argon. The reaction mixture was stirred at the same temperature for 30 min. The volatiles were removed under reduced pressure and the residue was dissolved in CH₂Cl₂ (80 mL), washed with aq. sodium metabisulfite solution and brine, dried over Na₂SO₄, concentrated, purified by flash column chromatography on silica gel (100% CH₂Cl₂ to 3% EtOAc/CH₂Cl₂) to give pure product (0.58 g, 89%). IR (neat, cm⁻¹) 1778, 1769, 1716; ¹H NMR (400 MHz, CDCl₃) δ 7.44-7.27 (m, 10H, Ar), 6.95 (s, 1H, CHPh₂), 4.97 (d, J=13.5 Hz, 1H, CH₂OAc), 4.90 (d, J=5.4 Hz, 1H, C6 CH), 4.74 (d, J=13.5 Hz, 1H, CH₂OAc), 3.57 (m, 1H, C7 CH), 3.58-3.35 (m, 4H, C2 CH and SCH₂), 2.01 (s, ¹³H, Ac); ¹³C NMR (100 MHz, CDCl₃) δ 184.0 (q, J=40.5 Hz, CF₃CO), 170.5, 163.0, 160.5, 139.1, 138.9, 128.5, 128.4, 128.2, 128.1, 127.6, 127.0, 126.3, 125.6, 115.2 (q, J=289 Hz, CF₃), 79.8, 62.8, 54.4, 52.8, 26.9, 23.5, 20.6.

### EXAMPLE 10

Benzhydryl 7β-(mercaptomethyl)cephalosporanate (11). To a solution of sodium acetate (0.29 g, 3.55 mmol) in acetic acid (0.20 mL, 3.55 mmol) and anhyd MeOH (8 mL) at 0° C. was added the solution of benzhydryl 7β-(trifluoroacetylsulfanylmethyl)cephalosporanate (10) (0.67 g, 1.18 mmol) in anhyd THF (4 mL) under argon. The reaction mixture was stirred at the same temperature for 30 min. The mixture was diluted with CH₂Cl₂ (100 mL), washed with cold water, water and brine, dried over Na₂SO₄, and concentrated. Further purification by flash column chromatography on silica gel (1% EtOAc/CH₂Cl₂ to 10% EtOAc/CH₂Cl₂) afforded pure product (0.5 g, 72%). IR (neat, cm⁻¹) 2567(SH), 1774, 1736, ¹H NMR (400 MHz, CDCl₃) δ 7.44-7.24 (m, 10H, Ar), 6.95 (s, 1H, CHPh₂), 4.91 (d, J=13.3 Hz, 1H, CH₂OAc), 4.89 (d, J=5.4 Hz, 1H, C6 CH), 4.68 (d, J=13.3 Hz, 1H, CH₂OAc), 3.89 (m, 1H, C7 CH), 3.51 (d, J=18.2 Hz, 1H, C2 CH), 3.31 (d, J=18.2 Hz, 1H, C2 CH), 2.93 (m, 2H, SCH₂), 1.98 (s, 3H, Ac), 1.79 (t, J=8.5 Hz, 1H, SH); ¹³C NMR (100 MHz, CDCl₃) δ 170.2, 164.0, 160.6, 139.0, 138.8, 128.3, 128.2, 128.0, 127.9, 127.5, 126.8, 126.3, 124.3, 79.5, 62.8, 57.5, 54.6, 26.6, 20.5, 18.8. HRMS(FAB) calcd for (M+Li) C₂₄H₂₃NO₅S₂Li 476.1178, found 476.1165.

### EXAMPLE 11

Sodium salt of 7β-(mercaptomethyl)cephalosporanate (12). To a solution of benzhydryl 7β-(mercaptomethyl)cephalosporanate (0.12 g, 0.255 mmol) in anhyd CH₂Cl₂ (2.4 mL) at −10° C. were added Et₃SiH (0.48 mL) and TFA (0.24 mL) slowly under argon. The reaction mixture was stirred at the same temperature for 10 min, the volatiles were removed under reduced pressure. The residue was dissolved in EtOAc (8 mL), extracted with aqueous NaHCO₃ (0.21 mg of NaHCO₃ was dissolved in 4 mL of deionied water). The separated aqueous layer was purified on a column of CHP20P (Mitsubishi Chemical Corporation) (Deionized water as eluent). The fractions were collected and lyophilized to give pure product (0.23 mg, 28%). ¹H NMR (400 MHz, D₂O) δ 4.95 (d, J=5.1 Hz, 1H, C6 CH), 4.71 (d, J=12.3 Hz, 1H, CH₂OAc), 4.54 (d, J=12.3 Hz, 1H, CH₂OAc), 3.88 (m, 1H, C7 CH), 3.51 (d, J=17.8 Hz, 2H, C2 CH), 3.23 (d, J=17.8 Hz, 2H, C2 CH), 2.84 (m, 2H, SCH₂), 1.96 (s, 3H, Ac).

### EXAMPLE 12

Benzhydryl 7β-(trifluoroacetylsulfanylmethyl)cephalosporanate-1,1-dioxide (13). To a solution of benzhydryl 7β-(trifluoroacetylsulfanylmethyl)-cephalosporanate-1β-oxide (8) (0.8 g, 1.37 mmol) in CH₂Cl₂ (14 mL) was added mCPBA (70%, 0.67 g, 2.75 mmol). After the reaction mixture was stirred at room temperature for 1 h, 0.67 g of m-CPBA was added. The mixture was continued to stir for 3 h, then diluted with CH₂Cl₂ (100 mL), washed with aqueous sodium metabisulfite, saturated NaHCO₃ solution and brine, dried over Na₂SO₄, evaporated to give almost pure product. It was utilized in the following reaction without further purification. IR (neat, cm⁻¹) 1794, 1737, 1716, 1216, 1165 (SO₂); ¹H NMR (400 MHz, CDCl₃) δ 7.45-7.28 (m, 10H, Ar), 6.93 (s, 1H, CHPh₂), 4.97 (d, J=14.1 Hz, 2H, CH₂OAc), 4.72 (d, J=5.1 Hz, 1H, C6 CH), 4.65 (d, J=14.1 Hz, 2H, CH₂OAc), 4.02 (m, 1H, C7 CH), 3.96 (d, J=18.6 Hz, 2H, C2 CH), 3.74 (m, 2H, SCH₂), 3.68 (d, J=18.6 Hz, 2H, C2 CH), 1.98 (s, 3H, Ac); ¹³C NMR (100 MHz, CDCl₃) δ 184.4 (q, J=40.2 Hz, CF₃CO), 170.1, 162.2, 159.5, 138.6, 138.5, 128.6, 128.5, 128.2, 128.1, 127.5, 126.9, 124.6, 124.1, 112 (q, J=289 Hz, CF₃), 80.6, 65.6, 61.9, 53.4, 51.5, 23.0, 20.4.

### EXAMPLE 13

Benzhydryl 7β-(mercaptomethyl)cephalosporanate-1,1-dioxide (14). Prepared from benzhydryl 7β-(trifluoroacetylsulfanylmethyl)cephalosporanate-1,1-dioxide (13) by the same method as utilized in the preparation of benzhydryl 7β-(mercaptomethyl)cephalosporanate (11) above (72%). IR (neat, cm⁻¹) 2569 (SH), 1791, 1730, 1228, 1131 (SO₂); ¹H NMR (400 MHz, CDCl₃) δ 7.44-7.25 (m, 10H, Ar), 6.95 (s, 1H, CHPh₂), 5.01 (d, J=14.1Hz, 1H, CH₂OAc), 4.76 (d, J=5.1 Hz, 1H, C6 CH), 4.66 (d, J=14.1Hz, 1H, CH₂OAc), 4.12 (m, 1H, C7 CH), 3.95 (d, J=18.5 Hz, 1H, C2 CH), 3.70 (d, J=18.5 Hz, 1H, C2 CH), 3.32-3.13 (m, 2H, SCH₂), 2.06 (t, J=9.0 Hz, 1H, SH), 2.01 (s, 3H, Ac); ¹³C NMR (100 MHz, CDCl₃) δ 170.2, 163.0, 159.6, 138.6, 138.5, 128.6, 128.5, 128.4, 128.3, 127.6, 127.0, 125.0, 123.3, 80.6, 66.3, 62.0, 59.5, 51.8, 20.5, 18.6. HRMS(FAB) calcd for (M+Li) C₂₄H₂₃NO₇S₂Li 508.1076, found 508.1072.

### EXAMPLE 14

Sodium salt of 7β-(mercaptomethyl)cephalosporanate-1,1-dioxide (15). A solution of benzhydryl 7β-(mercaptomethyl)cephalosporanate-1,1-dioxide (14) (0.112 g, 0.24 mmol) in m-cresol (0.9 mL) was heated at 50° C. for 3 h under an argon atmosphere. The mixture was then cooled to room temperature, diluted with ether (5 mL) and treated with aqueous NaHCO₃ solution (0.22 mg of NaHCO₃ was dissolved in 4 mL of deionied water, 0.26 mmol). The separated aqueous layer was then purified on a column of CHP20P (Mitsubishi Chemical Corporation) (Deionized water as eluent) and freeze dried to give pure 15 (64 mg, 75%). ¹H NMR (400 MHz, D₂O) δ 5.25 (d, J=5.0 Hz, 1H, C6 CH), 4.81 (d, J=12.8 Hz, 1H, CH₂OAc), 4.64 (d, J=12.8 Hz, 1H, CH₂OAc), 4.31 (m, 1H, C7 CH), 4.27 (d, J=17.5 Hz, 1H, C2 CH), 3.92 (d, J=17.5 Hz, 1H, C2 CH), 3.14 (m, 2H, SCH₂), 2.06 (s, 3H, Ac).

### EXAMPLE 15

Benzhydryl 7β-(mercaptomethyl)cephalosporanate-1β-oxide (16). Prepared from benzhydryl 7β-(trifluoroacetylsulfanylmethyl)cephalosporanate-1β-oxide (8) by the same method as utilized for the preparation of benzhydryl 7β-(mercaptomethyl)cephalosporanate (11) above (95%). IR (neat, cm⁻¹) 2565 (SH), 1781, 1736; ¹H NMR (400 MHz, CDCl₃) δ 7.47-7.26 (m, 10H, Ar), 6.97 (s, 1H, CHPh₂), 5.18 (d, J=13.6 Hz, 1H, CH₂OAc), 4.64 (d, J=13.6 Hz, 1H, CH₂OAc), 4.34 (d, J=4.9 Hz, 1H, C6 CH), 4.05-4.00 (m, 1H, C7 CH), 3.76 (d, J=18.5 Hz, 1H, C2 CH), 3.31-3.06 (m, 2H, SCH₂), 3.22 (d, J=18.5 Hz, 1H, C2 CH), 2.00 (s, 3H, Ac), 1.81-1.77 (dd, J=6.6 Hz, 1H, SH); ¹³C NMR (100 MHz, CDCl₃) δ 170.4, 163.4, 159.8, 139.0, 138.9, 128.5, 128.4, 128.2, 128.1, 127.7, 127.1, 126.1, 117.7, 80.3, 64.7, 63.5, 58.2, 46.7, 20.6, 18.3. HRMS(FAB) calcd for (M+Li) C₂₄H₂₃NO₆S₂Li 492.1127, found 492.1131.

### EXAMPLE 16

Sodium salt of 7β-(mercaptomethyl)cephalosporanate-1β-oxide (17). Prepared from benzhydryl 7β-(mercaptomethyl)cephalosparonate-1β-oxide (16) by the same method as utilized for the preparation of the sodium salt of 7β-(mercaptomethyl)cephalosporanate-1,1-dioxide (15) above (54%). ¹H NMR (400 MHz, D₂O) δ 4.86 (d, J=12.6 Hz, 1H, CH₂OAc), 4.75 (d, J=5.0 Hz, 1H, C6 CH), 4.54 (d, J=12.6 Hz, 1H, CH₂OAc), 4.20-4.10 (m, 1H, C7 CH), 3.75 (d, J=18.3 Hz, 1H, C2 CH), 3.51 (d, J=18.3 Hz, 1H, C2 CH), 3.03-2.95 (m, 2H, SCH₂), 1.95 (s, 3H, Ac).

### EXAMPLE 17

Benzhydryl 7α-(mercaptomethyl)cephalosporanate-1β-oxide (18). Prepared from benzhydryl 7α-(trifluoroacetylsulfanylmethyl)cephalosporanate-1β-oxide (9) by the same method as utilized in the preparation of benzhydryl 7β-(mercaptomethyl)cephalosporanate (11) above (85%). IR (neat, cm⁻¹) 2568 (SH), 1782, 1735; ⁻¹H NMR (400 MHz, CDCl₃) δ 7.47-7.25 (m, 10H, Ar), 6.92 (s, 1H, CHPh₂), 5.05 (d, J=13.5 Hz, 1H, CH₂OAc), 4.61 (d, J=13.5 Hz, 1H, CH₂OAc), 4.31 (s, 1H, C6 CH), 3.82 (bs, 1H, C7 CH), 3.68 (d, J=18.5 Hz, 1H, C2 CH), 3.22 (d, J=18.5 Hz, 1H, C2 CH), 2.96-2.89 (m, 2H, SCH₂), 1.97 (s, 3H, Ac), 1.60 (t, J=7.8 Hz, 1H, SH); ¹³C NMR (100 MHz, CDCl₃) δ 170.2, 162.2, 159.6, 138.9, 138.8, 128.3, 128.2, 128.0, 127.9, 127.4, 126.7, 125.9, 116.6, 80.0, 64.2, 62.8, 55.1, 46.0, 21.0, 20.4. HRMS (FAB) calcd for (M+Li) C₂₄H₂₃NO₆S₂Li 492.1127, found 492.1119.

### EXAMPLE 18

Sodium salt of 7α-(mercaptomethyl)cephalosporanate-1β-oxide (19). Prepared from benzhydryl 7α-(mercaptomethyl)cephalosporanate-1β-oxide (18) by the same method as utilized in the case of sodium salt of 7β-(mercaptomethyl)cephalosporanate-1,1-dioxide (15) above (33%). ¹H NMR (400 MHz, D₂O) δ 4.89 (d, J=12.6 Hz, 1H, CH₂OAc), 4.80 (d, J=1.7 Hz, 1H, C6 CH), 4.60 (d, J=12.6 Hz, 1H, CH₂OAc), 3.95 (d, J=18.2 Hz, 1H, C2 CH), 3.88-3.83 (m, 1H, C7 CH), 3.57 (d, J=18.2 Hz, 1H, C2 CH), 3.03 (d, J=6.0 Hz, 2H, SCH₂), 2.06 (s, 3H, Ac).

### EXAMPLE 19

Benzhydryl 7α-(trifluoroacetylsulfanylmethyl)cephalosporanate-1,1-dioxide (20). Prepared from benzhydryl 7α-(trifluoroacetylsulfanylmethyl)cephalospor-anate-1β-oxide (9) by the same method as utilized in the preparation of benzhydryl 7β-(trifluoroacetylsulfanylmethyl)cephalosporonate-1,1-dioxide (13) above (95%). IR (neat, cm⁻¹) 1797, 1736, 1716, 1218, 1167 (SO₂); ¹H NMR (400 MHz, CDCl₃) δ 7.45-7.27 (m, 10H, Ar), 6.95 (s, 1H, CHPh₂), 4.96 (d, J=14.0 Hz, 1H, CH₂OAc), 4.64 (s, 1H, C6 CH), 4.63 (d, J=14.0 Hz, 1 H, CH₂OAc), 4.12 (m, 1 H, C7 CH), 3.98 (d, J=18.3 Hz, 1H, C2 CH), 3.73 (d, J=18.3 Hz, 1H, C2 CH), 3.49 (m, 2H, SCH₂), 1.97 (s, 3H, Ac); ¹³C NMR (100 MHz, CDCl₃) δ 184.0 (q, J=40 Hz, CF₃CO), 170.1, 160.7, 159.4, 138.6, 138.5, 128.5, 128.4, 128.3, 128.1, 127.4, 126.8, 125.0, 124.8, 115.2 (q, J=289 Hz, CF₃), 80.4, 66.9, 61.6, 51.4, 50.6, 25.1, 20.3.

### EXAMPLE 20

Benzhydryl 7α-(mercaptomethyl)cephalosporanate-1,1-dioxide (21). Prepared from benzhydryl 7α-(trifluoroacetylsulfanylmethyl)cephalosporanate-1,1-dioxide (20) by the same method as utilized in the case of benzhydryl 7β-(mercaptomethyl)cephalosporanate (11) above (66%). IR (neat, cm⁻¹) 2585(SH), 1791, 1738, 1222, 1121(SO₂); ¹H NMR (400 MHz, CDCl₃) δ 7.44-7.26 (m, 10H, Ar), 6.90 (s, 1H, CHPh₂), 5.01 (d, J=14.1 Hz, 1H, CH₂OAc), 4.75 (s, 1H, C6 CH), 4.68 (d, J=14.1 Hz, 1H, CH₂OAc), 4.13 (m, 1H, C7 CH), 3.98 (d, J=18.2 Hz, 1H, C2 CH), 3.75 (d, J=18.2 Hz, 1H, C2 CH), 3.01-3.88 (m, 2H, SCH₂), 2.01 (s, 3H, Ac), 1.45(dd, J=7.8 Hz, 1H, SH); ¹³C NMR (100 MHz, CDCl₃) δ 17.02, 161.6, 159.4, 138.8, 138.7, 128.6, 128.5, 128.3, 128.2, 127.5, 127.0, 125.4, 124.9, 80.5, 66.4, 61.7, 54.8, 51.3, 20.9, 20.5. HRMS(FAB) calcd for (M+Li) C₂₄H₂₃NO₇S₂Li 508.1076, found 508.1059.

### EXAMPLE 21

Sodium salt of 7α-(mercaptomethyl)cephalosporanate-1,1-dioxide (22). Prepared from benzhydryl 7α-(mercaptomethyl)cephalosporanate-1,1-dioxide (21) by the same method as utilized in the case of sodium salt of 7β-(mercaptomethyl)-cephalosporanate-1,1-dioxide (15) above (76%). ¹H NMR (400 MHz, D₂O) δ 5.22 (s, 1H, C6 CH), 4.76 (d, J=12.7 Hz, 1H, CH₂OAc), 4.61 (d, J=12.7 Hz, 1H, CH₂OAc), 4.27 (d, J=17.1 Hz, 2H, C2 CH), 4.14 (m, 1H, C7 CH), 3.97 (d, J=17.1 Hz, 2H, C2 CH), 3.02 (m, 2H, SCH₂), 2.06 (s, 3H, Ac).

### EXAMPLE 22

Benzhydryl 7β-(trifluoroacetylsulfanylmethyl)cephalosporanate-1α-oxide (23) and Benzhydryl 7α-(trifluoroacetylsulfanylmethyl)cephalosporanate (24). To a solution of benzhydryl 7-methylenecephalosparate-1α-oxide (3.0 g, 6.61 mmol) in anhyd CH₂Cl₂ (45 mL) was added trifluorothioacetic acid (2.45 mL, 26.43 mmol) under argon. After the mixture was cooled to 0° C., pyridine (0.70 g, 0.71 mL, 8.81 mmol) was added slowly. The reaction was stirred at room temperature overnight. The volatiles were removed under reduced pressure, the residue was purified by flash column chromatography on silica gel (10% EtOAc/CH₂Cl₂ to 20% EtOAc/CH₂Cl₂) to give benzhydryl 7β-(trifluoroacetylsulfanylmethyl)-cephalosporanate-1α-oxide (23) (1.7 g, 44%) and benzhydryl 7α-(trifluoroacetyl-sulfanylmethyl)cephalosporanate (24) (1.0 g, 27%).

### EXAMPLE 23

Benzhydryl 7α-(trifluoroacetylsulfanylmethyl)cephalosporanate-1α-oxide (23) (top spot) IR (neat, cm⁻¹) 1789, 1739, 1716; ¹H NMR (400 MHz, CDCl₃) δ 7.42-7.26 (m, 10H, Ar), 6.93 (s, 1H, CHPh₂), 5.00 (d, J=14.0 Hz, 1H, CH₂OAc), 4.71 (d, J=14.0 Hz, 1H, CH₂OAc), 4.63 (d, J=4.9 Hz, 1H, C6 CH), 4.17 (m, 1H, C7 CH), 4.11 (d, J=17.2 Hz, 1H, C2 CH), 3.65 (d, J=8.1 Hz, 2H, SCH₂), 3.54 (d, J=17.2 Hz, 1H, C2 CH), 2.05 (s, 3H, Ac); ¹³C NMR (100 MHz, CDCl₃) δ 183.4 (q, J=41 Hz, CF₃CO), 170.0, 160.9, 159.2, 138.5, 138.4, 128.3, 128.2, 128.1, 128.0, 127.3, 126.6, 125.4, 123.5, 115.1 (q, J=289 Hz, CF₃), 80.2, 69.9, 61.7, 52.8, 49.4, 22.8, 20.1.

### EXAMPLE 24

Benzhydryl 7α-(trifluoroacetylsulfanylmethyl)cephalosporanate (24) (bottom spot) IR (neat, cm⁻¹) 1780, 1739, 1713; ¹H NMR (400 MHz, CDCl₃) δ 7.43-7.31 (m, 10H, Ar), 6.97 (s, 1H, CHPh₂), 4.93 (d, J=13.3 Hz, 1H, CH₂OAc), 4.71 (d, J=13.3 Hz, 1H, CH₂OAc), 4.51 (d, J=1.8 Hz, 1H, C6 CH), 4.17 (m, 1H, C7 CH), 3.57 (d, J=18.4 Hz, 1H, C2 CH), 3.54 (m, 2H, SCH₂), 3.35 (d, J=18.4 Hz, 1H, C2 CH), 2.00 (s, 3H, Ac); ¹³C NMR (100 MHz, CDCl₃) δ 184.1 (q, J=41 Hz, CF₃CO), 170.4, 161.5, 160.5, 139.2, 138.9, 128.5, 128.4, 128.2, 128.1, 127.7, 127.0, 126.8, 123.6, 115.4 (q, J=289 Hz, CF₃), 79.9, 62.8, 57.2, 53.9, 28.1, 26.4, 20.6.

### EXAMPLE 25

Benzhydryl 7α-(mercaptomethyl)cephalosporanate (25). Prepared from benzhydryl 7α-(trifluoroacetylsulfanylmethyl)cephalosporanate (24) by the same method as utilized to prepare benzhydryl 7β-(mercaptomethyl)cephalosporanate (11) above (69%). IR (neat, cm⁻¹) 2568 (SH), 1777, 1737; ¹H NMR (400 MHz, CDCl₃) δ 7.46-7.25 (m, 10H, Ar), 6.95 (s, 1H, CHPh₂), 4.92 (d, J=13.2 Hz, CH₂OAc), 4.70 (d, J=13.2 Hz, 1H, CH₂OAc), 4.61 (d, J=2.2 Hz, 1H, C6 CH), 3.43 (m, 1H, C7 CH), 3.33 (d, J=18.3 Hz, 1H, C2 CH), 3.00-2.94 (m, 2H, SCH₂), 1.99 (s, 3H, Ac), 1.57 (t, J=8.6 Hz, 1H, SH); ¹³C NMR (100 MHz, CDCl₃) δ 170.4, 162.7, 160.6, 139.2, 139.0, 128.4, 128.3, 128.1, 127.9, 127.6, 126.9, 122.8, 79.7, 62.8, 60.7, 53.0, 28.1, 21.8, 20.6. HRMS (FAB) calcd for (M+Li) C₂₄H₂₃NO₅S₂Li 476.1178, found 476.1175.

### EXAMPLE 26

Sodium salt of 7α-(mercaptomethyl)cephalosporanate (26). Prepared from benzhydryl 7α-(mercaptomethyl)cephalosporanate (25) by the same method as utilized in the case of sodium salt of 7β-(mercaptomethyl)cephalosporanate (12) above (yield=15%). ¹H NMR (400 MHz, D₂O) δ 4.86 (d, J=12.3 Hz, 1H, CH₂OAc), 4.74 (d, J=1.7 Hz, 1H, C6 CH), 4.66 (d, J=12.3 Hz, 1H, CH₂OAc), 3.64 (d, J=17.7 Hz, 1H, C2 CH), 3.58-3.55 (m, 1H, C7 CH), 3.34 (d, J=17.7 Hz, 1H, C2 CH), 3.20-2.90 (m, 2H, SCH₂), 2.09 (s, 3H, Ac).

### EXAMPLE 27

Benzhydryl 7β-(trifluoroacetylsulfanylmethyl)cephalosporanate-1α-oxide (27). To a solution of benzhydryl 7α-(trifluoroacetylsulfanylmethyl)-cephalosporanate (24) (1.0 g, 1.77 mmol) in CH₂Cl₂ (20 mL) was added m-CPBA (70%, 0.43 g, 1.77 mmol). The reaction mixture was stirred at room temperature for 1 h. The reaction mixture was diluted with CH₂Cl₂ (100 mL), washed with aq. sodium metabisulfite solution, saturated NaHCO₃ solution and brine, evaporated, the residue was purified by flash column chromatography on Silica Gel (15% EtOAc/CH₂Cl₂ to 40% EtOAc/CH₂Cl₂) to give α sulfoxide, 27, (0.23 g, 22%) and β sulfoxide, 9, (0.66g, 64%).

Benzhydryl 7α-(trifluoroacetylsulfanylmethyl)cephalo-sporanate-1α-oxide (27) (top spot) IR (neat, cm⁻¹) 1787, 1736; ¹H NMR (400 MHz, CDCl₃) δ 7.45-7.26 (m, 10H, Ar), 6.94 (s, 1H, CHPh₂), 4.97 (d, J=14.9 Hz, 1H, CH₂OAc), 4.68 (d, J=14.9 Hz, 1H, CH₂OAc), 4.25 (s, 1H, C6 CH), 3.93-3.80 (m, 1H, C7 CH), 3.90 (d, J=17.0 Hz, 1H, C2 CH), 3.61-3.48 (m, 2H, SCH₂), 3.57 (d, J=17.0 Hz, 1H, C2 CH), 2.00 (s, 3H, Ac); ¹³C NMR (100 MHz, CDCl₃) δ 184.0 (q, J=40 Hz, CF₃CO), 170.0, 160.2, 159.2, 138.6, 138.5, 128.4, 128.3, 128.2, 128.1, 127.4, 126.8, 126.0, 124.4, 115.2 (q, J=289 Hz, CF₃), 80.1, 70.3, 61.8, 55.4, 50.3, 25.8, 20.3.

### EXAMPLE 28

Benzhydryl 7α-(mercaptomethyl)cephalosporanate-1α-oxide (28). Prepared from benzhydryl 7α-(trifluoroacetylsulfanylmethyl)cephalosporanate-1β-oxide by the same method as utilized for the preparation of benzhydryl 7β-(mercaptomethyl)cephalosporanate (11) above (42%). IR (neat, cm⁻¹) 2568 (SH), 1785, 1736; ¹H NMR (400 MHz, CDCl₃) δ 7.46-7.28 (m, 10H, Ar), 6.91 (s, 1H, CHPh₂), 4.99 (d, J=13.8 Hz, 1H, CH₂OAc), 4.72 (d, J=13.8 Hz, 1H, CH₂OAc), 4.42 (d, J=2.1 Hz, 1H, C6 CH), 3.92-3.89 (m, 1H, C7 CH), 3.98 (d, J=17.2 Hz, 1H, C2 CH), 3.15-2.90 (m, 2H, SCH₂), 3.53 (d, J=17.2 Hz, 1H, C2 CH), 2.03 (s, 3H, Ac), 1.57-1.53 (dd, J=7.8 Hz, 1H, SH); ¹³C NMR (100 MHz, CDCl₃) δ 170.1, 161.1, 159.3, 138.8, 138.7, 128.5, 128.4, 128.2, 128.3, 127.5, 126.9, 126.2, 123.8, 80.3, 69.4, 61.9, 60.0, 50.7, 21.4, 20.5. HRMS(FAB) calcd for (M+Li) C₂₄H₂₃NO₆S₂Li 492.1127,found 492.1130.

### EXAMPLE 29

Sodium salt of 7α-(mercaptomethyl)cephalosporanate-1α-oxide (29). Prepared from benzhydryl 7α-(mercaptomethyl)cephalosporanate-1β-oxide by the same method as utilized for the preparation of the sodium salt of 7β-(mercaptomethyl)cephalosporanate-1,1-dioxide above (43%). ¹H NMR (400 MHz, D₂O) δ 4.76 (d, J=12.4 Hz, 1H, CH₂OAc), 4.67 (d, J=1.8 Hz, 1H, C6 CH), 4.59 (d, J=12.4 Hz, 1H, CH₂OAc), 4.18 (d, J=16.1 Hz, 1H, C2 CH), 4.15-4.10 (m, 1H, C7 CH), 4.65 (d, J=16.1 Hz, 1H, C2 CH), 3.10-2.97 (m, 2H, SCH₂), 2.05 (s, 3H, Ac).

### EXAMPLE 30

The following illustrate representative pharmaceutical dosage forms, containing a compound of formula I (‘Compound X’), for therapeutic or prophylactic use in humans.

| (i) Tablet 1 | mg/tablet | |
|---|---|---|
| ‘Compound X’ | 100.0 | |
| Lactose | 77.5 | |
| Povidone | 15.0 | |
| Croscarmellose sodium | 12.0 | |
| Microcrystalline cellulose | 92.5 | |
| Magnesium stearate | 3.0 | |
| | 300.0 | |
| (ii) Tablet 2 | mg/tablet | |
| ‘Compound X’ | 20.0 | |
| Microcrystalline cellulose | 410.0 | |
| Starch | 50.0 | |
| Sodium starch glycolate | 15.0 | |
| Magnesium stearate | 5.0 | |
| | 500.0 | |
| (iii) Capsule | mg/capsule | |
| ‘Compound X’ | 10.0 | |
| Colloidal silicon dioxide | 1.5 | |
| Lactose | 465.5 | |
| Pregelatinized starch | 120.0 | |
| Magnesium stearate | 3.0 | |
| | 600.0 | |
| (iv) Injection 1 (1 mg/ml) | mg/ml | |
| ‘Compound X’ (free acid form) | 1.0 | |
| Dibasic sodium phosphate | 12.0 | |
| Monobasic sodium phosphate | 0.7 | |
| Sodium chloride | 4.5 | |
| 1.0 N Sodium hydroxide solution | q.s. | |
| (pH adjustment to 7.0-7.5) | | |
| Water for injection | q.s. ad 1 | mL |
| (v) Injection 2 (10 mg/ml) | mg/ml | |
| ‘Compound X’ (free acid form) | 10.0 | |
| Monobasic sodium phosphate | 0.3 | |
| Dibasic sodium phosphate | 1.1 | |
| Polyethylene glycol 400 | 200.0 | |
| 01 N Sodium hydroxide solution | q.s. | |
| (pH adjustment to 7.0-7.5) | | |
| Water for injection | q.s. ad 1 | mL |
| (vi) Aerosol | mg/can | |
| ‘Compound X’ | 20.0 | |
| Oleic acid | 10.0 | |
| Trichloromonofluoromethane | 5,000.0 | |
| Dichlorodifluoromethane | 10,000.0 | |
| Dichlorotetrafluoroethane | 5,000.0 | |
| (vii) Tablet | mg/tablet | |
| ‘Compound X’ | 100.0 | |
| β-lactam antibiotic | 100.0 | |
| Lactose | 77.5 | |
| Povidone | 15.0 | |
| Croscarmellose sodium | 12.0 | |
| Microcrystalline cellulose | 92.5 | |
| Magnesium stearate | 3.0 | |
| | 400.0 | |
| (viii) Tablet | mg/tablet | |
| ‘Compound X’ | 20.0 | |
| β-lactam antibiotic | 10.0 | |
| Microcrystalline cellulose | 410.0 | |
| Starch | 50.0 | |
| Sodium starch glycolate | 15.0 | |
| Magnesium stearate | 5.0 | |
| | 510.0 | |
| (ix) Capsule | mg/capsule | |
| ‘Compound X’ | 10.0 | |
| β-lactam antibiotic | 10.0 | |
| Colloidal silicon dioxide | 1.5 | |
| Lactose | 465.5 | |
| Pregelatinized starch | 120.0 | |
| Magnesium stearate | 3.0 | |
| | 610.0 | |
| (x) Injection 1 (1 mg/ml) | mg/ml | |
| ‘Compound X’ (free acid form) | 1.0 | |
| β-lactam antibiotic | 1.0 | |
| Dibasic sodium phosphate | 12.0 | |
| Monobasic sodium phosphate | 0.7 | |
| Sodium chloride | 4.5 | |
| 1.0 N Sodium hydroxide solution | q.s. | |
| (pH adjustment to 7.0-7.5) | | |
| Water for injection | q.s. ad 1 | mL |

The above formulations may be obtained by conventional procedures well known in the pharmaceutical art. The β-lactam antibiotic in the above formulations can be any β-lactam antibiotic, including those identified specifically hereinabove.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed:

1. A compound of formula (I)
See Formula I in the Figures Section
(I)
wherein
R¹ is hydrogen, (C₁-C₁₀)alkanoyl C(═O)R, (C₁-C₁₀)alkoxycarbonyl C(═O)OR, (C₁-C₁₀)carboxamido (C═O)NHR, (C₁-C₁₀)alkyl, (C₃-C₈)cycloalkyl, (C₂-C₁₀)alkenyl, (C₂-C₁₀)alkynyl, aryl, heteroaryl, oxazolidinyl, isoxazolidinyl, or morpholinyl;
R² is hydrogen, (C₁-C₁₀)alkyl, (C₃-C₈)cycloalkyl, (C₂-C₁₀)alkenyl, (C₂-C₁₀)alkynyl, —COOR, —CONRR, cyano, —C(═O)R, aryl, heteroaryl, oxazolidinyl, isoxazolidinyl, morpholinyl, —S(O)ₙR, —NRR, azido, or halo;
R³ is acetoxymethyl (CH₂OAc), carbamoyloxymethyl (CH₂O(C═O)NH₂), hydrogen, methyl, halo, (C2-C10)alkyl, cyano, vinyl (—CH═CH2), (C1-C10)substituted vinyl (—CH═CHR), (C1-C10)alkanoylvinyl (—CH═CHCOR), (C1-C10)alkoxycarbonylvinyl (—CH═CHCO2R), (C1-C10)carboxaminovinyl (—CH═CHCONHR), (C2-C10)alkynyl, (C1-C10)alkanoyl, (C3-C8)cycloalkyl, aryl, heteroaryl, (C1-C10)substituted aryl, (C1-C10)alkyl, heteroaryl(C1-C10)alkyl, S(O)nR wherein n=0, 1, or 2, corresponding to the C3 sidechain sulfide, sulfoxide and sulfone and R is (C1-C10)alkyl, aryl, substituted aryl, heteroaryl, substituted heteroaryl.
R⁴ is hydrogen, acyloxymethyl (CH2O(C═O)R), (C1-C10)alkyl, (C₃-C₈)cycloalkyl, (C₂-C₁₀)alkenyl, (C₂-C₁₀)alkynyl, aryl, or heteroaryl; or a pharmaceutically acceptable salt thereof, especially including sodium, potassium, and lithium.
n is 0, 1, or 2;

2. The synergistic combination of a compound of Formula I with a β-lactam antibiotic, including, but not limited to, members of the penicillin, cephalosporin, carbapenem, carbacephem, oxacephem, penem, and monobactam subcategories.
